(19) United States
(12) Patent Application Publication
(10) Pub. No.: US 2023/0225949 A1
(43) Pub. Date: Jul. 20, 2023

(54) Fermented Composition for Topical Application to Skin

(71) Applicant: Lynn E. Nguyen, Los Angeles, CA (US)

(72) Inventor: Lynn E. Nguyen, Los Angeles, CA (US)

(21) Appl. No.: 17/576,804

(22) Filed: Jan. 14, 2022

(51) Int. Cl. A61K 8/36; A61Q 19/00; A61K 8/92; A61K 8/34; A61K 8/9789; A61K 8/04

(52) U.S. Cl. CPC A61K 8/36; A61Q 19/00; A61K 8/92; A61K 8/345; A61K 8/9789; A61K 8/04

(57) ABSTRACT

A composition configured for topical application to the skin and formulated to reduce itching and inflammation is provided. The composition comprises a solution of vinegar, alcohol, and vegetable glycerin together with certain combinations of raw plant materials selected from rosemary, lavender, lemongrass, bergamot, aloe vera, ginger root, roman chamomile, nutmeg, parsley leaf, orange peel, and grapefruit peel. The solution including raw plant material may be periodically stirred over a period of weeks or months to mix ingredients and facilitate fermentation and microorganism activity. When applied to the skin, the nutrient density of beneficial microorganisms on the skin, including certain biproducts of the fermentation, may be increased to improve the skins acceptance and absorption of such microorganisms and byproducts.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to topical skin treatments, and more particularly to fermented formulations of raw plant material for reducing itching and inflammation.

### BACKGROUND

Skin is the largest organ of the human body and shields a majority of the body's other organs from damage and infection caused by bacteria, fungus, viruses, and other external elements. Although the skin itself is largely protected by a large community of beneficial microorganisms that live on its upper surface, the skin remains susceptible to its own infections caused by pathogens and other disorders, which can lead to immune responses or otherwise present as uncomfortable, itchy, and even unsightly inflammation, scaling, lesions, and rashes.

A number of pharmaceutical options have been introduced in the marketplace to address skin inflammation, such as, corticosteroid creams, antihistamines, and hydrocortisone. These only address the skin inflammation as a symptom of immune response, though, rather than the cause of the response itself. For instance, corticosteroid creams and hydrocortisone suppress the immune system in order reduce inflammation, rather than ridding the body of the bacteria, virus, or fungus causing the immune response. As another example, antihistamines block the body from releasing histamines as an immune response meant to remove seemingly threatening allergens from the body. Thus, while these attempt to reduce the uncomfortable redness, heat, itching, sensitivity and swelling that may accompany skin inflammation as a symptom of some infections or other conditions, they ultimately fail to treat the cause.

Even as these proposed pharmaceutical compositions have been made readily available to consumers, consumers are becoming increasingly judicious about the products they are putting in and on their bodies. Indeed, many expressly seek out alternate and seemingly more “natural” methods for addressing medical concerns, such as essential oils, to provide relief. Unfortunately, scientific evidence does not currently indicate that essential oils are effective in treating the cause or even symptoms of skin inflammation and rashes.

One method of naturally addressing the cause of inflammation and immune response internally has been to cultivate beneficial microorganisms, such as “probiotics”, through the fermentation of certain foods and then introduce such microorganisms to the intestinal tract by way of digestion. Some have similarly proposed topically applying fermented products to the skin. More particularly, examples or proposed uses of fermentation topical products include the following patent documents, incorporated herein by reference: Japanese Patent No. 5,467,106 disclosing the use of fermented mung beans for anti-aging; Korean Patent No. 101,333,130 disclosing the use of fermented rice and citrus to promote hair growth; Korean Patent Application No. 2012/0068367 disclosing low temperature fermentation for use with antioxidation, anti-wrinkling, whitening, and moisturization; and U.S. Pat. No. 10,406,097 to Thorel, teaching the use of bacteria on damage caused by UV light and to fight cell aging.

Although various solutions provide treatments for rashes and skin inflammation, none combine the characteristics of the present invention. Thus, there remains a need for a natural formulation to address the cause of skin rashes, itching, and/or inflammation.

### SUMMARY

The present disclosure is directed to a composition for topical application to treat uncomfortable and even unsightly skin conditions including, without limitation, itching and inflammation, from any number of causes, such as insect bites and medical conditions including psoriasis.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In an embodiment, the composition comprises one or more raw plant materials selected from the following species: rosemary, lavender, lemongrass, bergamot, aloe vera, ginger root, roman chamomile, nutmeg, parsley leaf, orange and/or orange peel, and grapefruit and/or peel. In addition, the composition comprises vinegar, alcohol, and glycerin. The glycerin may be pure vegetable glycerin. It is contemplated that the raw plant materials may be mixed in a solution with the vinegar, alcohol, and glycerin, and the solution and then sealed in a container together in a manner that facilitates anaerobic fermentation. In some embodiments, the combined ingredients may be periodically stirred, mixed, or otherwise agitated to facilitate microorganism activities and processes further facilitating anaerobic fermentation. Such mixing, stirring, or agitation may occur on an hourly, daily, weekly, or even monthly basis.

In some embodiments, the ingredients may remain sealed for a period of weeks or months, or until a desirable level of microorganism activity or production has been met. Additionally, the mixed ingredients may be maintained between about 60 and about 80 degrees Fahrenheit in order to support microorganism activity.

It is contemplated that combining ingredients in this manner and subjecting them to anaerobic fermentation may result in the transformation of raw materials or production of byproducts that may have desired beneficial health effects, such as the reduction of skin itching and inflammation, among others. In some embodiments these beneficial byproducts may comprise, for example only and not limitation, free fatty acids, mannosylerythrtol lipids (MELs), antioxidants, peptides, enzymes, and acids, such as alpha-hydroxy acids, lactic acid, and hyaluronic acid.

It is contemplated that formulating a composition for topical application according to the disclosure and claims provided herein may have the following advantages:
a) it may utilize natural and raw materials;
b) it may increase the population of beneficial microorganisms on the skin's surface;
c) it may supplant the population of undesirable or potentially damaging or otherwise unhealthy microorganisms on the skin's surface;
d) it may treat certain causes of skin irritation, rashes, inflammation, and other conditions; and
e) it may reduce certain symptoms of skin conditions.

Thus, it is an object of the invention to avoid the introduction of harmful chemicals to the users' skin by utilizing natural and raw materials to address various skin conditions.

It is another object of the invention to contribute to a healthy skin biome through the introduction of anaerobically cultivated microorganisms.

It is still another object of the invention to increase the population of beneficial microorganisms on the skin and makes such beneficial microorganisms, rendering a greater number of beneficial microorganisms available for absorption through the skin.

It is yet another object of the invention to treat certain causes of rashes, irritation, inflammation, and other skin conditions by supplanting the population of potentially harmful or undesirable microorganisms on the skin.

It is another object of the invention to relieve and/or reduce symptoms associated with rashes, irritation, inflammation, and other skin conditions, such as uncomfortable and/or unsightly itching, welts, rashes, heat, scaling, and flaking, among others.

One or more of the above-disclosed embodiments, in addition to certain alternatives, are provided in further detail below with reference to the attached figures. The disclosed subject matter is not, however, limited to any particular embodiment disclosed.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

In accordance with one embodiment, the composition may comprise between about 20 to 70 weight percent acetic acid, between about 15 to 60 weight percent raw plant materials, between about 1 to 15 weight percent alcohol, and between about 1 to 15 weight percent glycerin.

In another embodiment, the composition may comprise between about 40 to 60 weight percent acetic acid, between about 20 to 50 weight percent raw plant materials, between about 3 to 10 weight percent alcohol, and between about 3 to 7 weight percent glycerin.

In yet another embodiment, the composition may comprise about 50 to 60 weight percent acetic acid, between about 20 to 40 weight percent raw plant materials, between about 3 to 7 weight percent alcohol, and between about 3 to 7 weight percent glycerin.

In one exemplary embodiment, the acetic acid may be vinegar. In addition, the glycerin, alternatively known as glycerine and glycerol, may be pure vegetable glycerin. It is further contemplated that the raw plant materials may be rosemary, lavender, lemongrass, bergamot, aloe vera, ginger root, roman chamomile, nutmeg, parsley leaf, orange, grapefruit, and/or orange and/or grapefruit peels, or a combination thereof. In such an embodiment, the composition may include about 0.5 to about 3 weight percent rosemary, about 0.5 to about 3 weight percent lavender, about 5 to about 10 weight percent lemongrass, about 2 to about 6 weight percent bergamot, about 1 to about 5 weight percent aloe vera, about 0.5 to about 3 weight percent ginger root, about 5 to 10 weight percent roman chamomile, less than about 3 percent nutmeg or nutmeg powder, about 0.5 to about 3 weight percent parsley leaf, about 3 to about 8 percent orange peel, and about 3 to about 8 percent grapefruit peel.

In an embodiment, the composition may be fermented. It is further envisioned that the fermented composition will contain microorganisms. In one embodiment, these microorganisms are applied topically to skin. In such an embodiment, the microorganisms are applied to a user's skin to recolonize, supplant, or support the skin's microbiome.

It will be understood that the process of fermenting the composition may make use of a natural process to transform raw materials into new byproducts. In an embodiment, the composition is fermented by placing acetic acid, raw plant materials, alcohol, and glycerin in a sealable container. Sealing the container may enable anaerobic microorganism activity. The contents of the container may be periodically mixed to aid with such activity. In such an embodiment, the fermentation may occur over a period of weeks or several months. Of course, it should be noted that other processing techniques known to those of ordinary skill in the art may be implemented to achieve similar outcomes.

In some embodiments, solid matter may be strained out of the fermented composition prior to applying the composition to the skin. That is, in the event that raw plant material fails to dissolve over the course of fermentation, such raw plant material may be strained from the fermented solution to provide a liquid, gel, or other non-solid composition configured for topical application to a user's skin. In some instances, the raw plant material may be additionally ground or otherwise prepared in a manner that may maximize the amount of raw plant material that dissolves into the solution over the course of fermentation.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### EXAMPLES OF THE PREFERRED EMBODIMENT OF THE COMPOSITION

In order to more fully teach what the Applicant regards as its invention, the following example is given. It should be understood that the formulation set forth in the Example is not to be construed as limiting of the scope of the invention, except so far as it yields a topical formulation having the desired properties and characteristics. More particularly, and by way of example, the following chart illustrates a formulation of the same with the percentages given by weight of the composition.

### Example 1

| Ingredient | Percentage |
|---|---|
| Vinegar | 56.0 |
| Roman chamomile | 6.0 |
| Lemongrass | 6.0 |
| Orange peel | 5.0 |
| Grapefruit peel | 5.0 |
| Alcohol | 5.0 |
| Bergamot | 4.0 |
| Pure vegetable glycerin | 4.0 |
| Aloe vera | 3.0 |
| Ginger root | 2.0 |
| Lavender | 2.0 |
| Rosemary | 1.0 |
| Parsley leaf | 1.0 |
| TOTAL: | 100.00% |

### CONCLUSIONS, RAMIFICATIONS, AND SCOPE

While certain embodiments of the invention have been illustrated and described, various modifications are contemplated and can be made without departing from the spirit and scope of the invention. Accordingly, it is intended that the invention not be limited, except as by the appended claim(s).

The teachings disclosed herein may be applied to other compositions or systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the fermented composition for topical application to skin with which that terminology is associated. In general, the terms used in the following claims should not be constructed to limit the fermented composition for topical application to skin to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the fermented composition for topical application to skin is not intended to be exhaustive or limited to the precise form disclosed above or to a particular field of usage.

While specific embodiments of, and examples for, the composition are described above for illustrative purposes, various equivalent modifications are possible for which those skilled in the relevant art will recognize.

While certain aspects of the composition disclosed are presented below in particular claim forms, various aspects of the composition are contemplated in any number of claim forms. Thus, the inventor reserves the right to add additional claims after filing the application to pursue such additional claim forms for other aspects of the fermented composition for topical application to skin.

1. A composition comprising:
a. acetic acid;
b. at least one raw plant material selected from the group consisting of rosemary, lavender, lemongrass, bergamot, aloe vera, ginger root, roman chamomile, nutmeg, parsley leaf, orange, and grapefruit;
c. alcohol; and
d. glycerin.

2. The composition of claim 1, wherein the raw plant material is selected from the group consisting of rosemary, lavender, lemongrass, bergamot, aloe vera, ginger root, roman chamomile, nutmeg, parsley leaf, orange, and grapefruit.

3. The composition of claim 1, wherein the glycerin is pure vegetable glycerin.

4. The composition of claim 1, wherein the composition comprises up to about 4 percent by weight vegetable glycerin.

5. The composition of claim 1, wherein the composition comprises up to about 56 percent by weight vinegar.

6. The composition of claim 1, wherein the composition comprises up to about 5 percent by weight alcohol.

7. The composition of claim 1, wherein the composition comprises:
a. about 20 to 70 percent by weight acetic acid;
b. about 15 to 60 percent by weight raw plant material;
c. about 1 to 15 percent by weight alcohol; and
d. about 1 to 15 percent by weight glycerin.

8. The composition of claim 1, wherein the composition comprises:
a. about 40 to 60 percent by weight acetic acid;
b. about 20 to 50 percent by weight raw plant material;
c. about 3 to 10 percent by weight alcohol; and
d. about 3 to 7 percent by weight glycerin.

9. The composition of claim 1, wherein the composition comprises:
a. about 50 to 60 percent by weight acetic acid;
b. about 20 to 40 percent by weight raw plant material;
c. about 3 to 7 percent by weight alcohol; and
d. about 3 to 7 percent by weight glycerin.

10. The composition of claim 1, wherein the composition comprises:
a. up to about 6 percent by weight lemongrass;
b. up to about 5 percent by weight orange peel;
c. up to about 5 percent by weight grapefruit peel;
d. up to about 4 percent by weight bergamot;
e. up to about 3 percent by weight aloe vera;
f. up to about 2 percent by weight ginger root;
g. up to about 2 percent by weight lavender;
h. up to about 1 percent by weight rosemary; and
i. up to about 1 percent by weight parsley leaf.

11. The composition of claim 1, wherein the composition is fermented.

12. The composition of claim 1, wherein the composition is configured for application to the skin.

13. The composition of claim 1, wherein the composition is in the form of a lotion, cream, or gel.

14. A fermented composition for topical application to the skin formulated to reduce itching and inflammation, said composition comprising:
a. vinegar;
b. raw plant materials wherein the raw plant materials consist of rosemary, lavender, lemongrass, bergamot, aloe vera, ginger root, roman chamomile, nutmeg, parsley leaf, orange, grapefruit, or a combination thereof;
c. alcohol; and
d. pure vegetable glycerin.

15. The composition of claim 14, wherein the composition comprises from:
a. about 50 to 60 percent by weight vinegar;
b. about 20 to 40 percent by weight raw plant material;
c. about 3 to 7 percent by weight alcohol; and
d. about 3 to 7 percent by weight vegetable glycerin.

17. The composition of claim 14, comprising, as the raw plant material,
a. up to about 6 percent by weight roman chamomile;
b. up to about 6 percent by weight lemongrass;
c. up to about 5 percent by weight orange peel;
d. up to about 5 percent by weight grapefruit peel;
e. up to about 4 percent by weight bergamot;
f. up to about 3 percent by weight aloe vera;
g. up to about 2 percent by weight ginger root;
h. up to about 2 percent by weight lavender;
i. up to about 1 percent by weight rosemary; and
j. up to about 1 percent by weight parsley leaf.

18. The composition of claim 17, further comprising:
a. up to about 56 percent by weight vinegar;
b. up to about 5 percent by weight alcohol; and
c. up to about 4 percent by weight vegetable glycerin.

19. The composition of claim 15, wherein the composition is in the form of a lotion, cream, or gel.
